(19) United States
(12) Patent Application Publication
(10) Pub. No.: US 2003/0113832 A1
(43) Pub. Date: Jun. 19, 2003

(54) Apparatus and method for assaying electrophysiological effects

(76) Inventor: Robert J. Lauf, Oak Ridge, TN (US)

Correspondence Address: GRAY, CARY, WARE & FREIDENRICH LLP, 1221 SOUTH MOPAC EXPRESSWAY, SUITE 400, AUSTIN, TX, 78746-6875, US

(21) Appl. No.: 10/017,511

(22) Filed: Dec. 14, 2001

(51) Int. Cl. C12Q 1/02; C12M 1/34

(52) U.S. Cl. 435/29; 435/287.1

(57) ABSTRACT

A culture vessel for biological samples contains a plurality of individually addressable electrodes and a means of maintaining these electrodes at various potentials, whereby cells may be cultured in a wide variety of electric fields to rapidly assess the influence of field strength, frequency, or waveform on cell growth or inhibition, platelet adhesion, gene expression, or other responses of interest while maintaining an otherwise uniform culture environment for the cells. The electrode array may be formed on a rigid, reusable substrate or it may be disposable.

### STATEMENT AS TO RIGHTS TO INVENTIONS MADE UNDER FEDERALLY-SPONSORED RESEARCH AND DEVELOPMENT

This invention was made with United States Government support under Contract No. DE-AC05-00OR22725 awarded by the United States Department of Energy. The Government has certain rights in this invention.

### BACKGROUND OF THE INVENTION

1. Field of the Invention

The invention relates generally to the field of cellular biology. More particularly, the preferred embodiments of the invention relate to selectively varying the electric fields applied to a biological sample so the effects of electric field on the living cells may be evaluated.

2. Background of the Invention

In the field of cell biology, it is well known that externally-applied electric fields can influence biological parameters such as the healing of fractured bone. However, the mechanisms by which these effects take place are not well understood, and the determination of what electric fields might be optimal for a given biological problem are very time-consuming, since the researcher needs to eliminate other variables as much as possible. What is needed is an approach by which biological samples may be subjected to a large number of different, well-characterized electric fields while holding other environmental factors constant.

### SUMMARY OF THE INVENTION

There is a need for the following embodiments. Of course, the invention is not limited to these embodiments.

It is an object of this invention to provide a biological culture apparatus in which a sample may be subjected to a controlled application of various electric fields. It is another object of this invention to provide a biological culture apparatus in which a sample may be subjected to a controlled application of various electric fields while maintaining an otherwise uniform environment. It is another object of the present invention to provide a sterilizable, inert electrode array for subjecting a biological sample to a plurality of electric fields at a plurality of points, whereby the biological effects of these electric fields may be determined. It is another object of the present invention to provide a sterilizable, inert electrode array for subjecting different parts of a biological sample to a plurality of oscillating electrical signals having a plurality of waveforms, whereby the biological effects of these electric fields may be determined. It is another object of the present invention to provide a method for subjecting different parts of a biological sample to a plurality of oscillating electrical signals having a plurality of waveforms, whereby the optimal waveform for enhancing a desired biological effect may be determined. It is another object of this invention to provide a biological culture apparatus in which a sample may be subjected to a controlled application of various electric fields at selected points in an otherwise uniform environment while assaying the effect of the various electric fields on gene expression at the selected points. It is another object of this invention to provide an apparatus in which one biological sample may be subjected to various electrical signals in different locations simultaneously, whereby the cellular responses at these locations may be related to the fields applied at these locations because the entire culture is maintained in an otherwise common environment. It is another object to provide a method of assessing the effect of electric fields on cellular responses by culturing cells in a common environment and applying different electrical signals to the cells at various locations while assaying the cellular response to these signals.

Other objects and advantages will be accomplished by the present invention, which comprises a sterile culture vessel for biological samples, containing a plurality of individually addressable electrodes and a means of maintaining these electrodes at various potentials, whereby cells may be cultured in a wide variety of electric fields. The inventive device allows the researcher to rapidly assess the influence of field strength, frequency, or waveform on cell growth or inhibition, platelet adhesion, gene expression, or other responses of interest. Some applicable processes include cell growth and healing, scar formation, osteoclast and osteoblast behavior, seed germination, bacterial growth or inhibition, production of proteins or hormones, inflammatory responses, and uptake of pharmaceuticals.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The drawings accompanying and forming part of this specification are included to depict certain aspects of the invention. A clearer conception of the invention, and of the components and operation of systems provided with the invention, will become more readily apparent by referring to the exemplary, and therefore nonlimiting, embodiments illustrated in the drawings, wherein like reference numerals (if they occur in more than one view) designate the same elements. The invention may be better understood by reference to one or more of these drawings in combination with the description presented herein. It should be noted that the features illustrated in the drawings are not necessarily drawn to scale.

FIG. 1 is a schematic diagram, shown in cross-section, of one preferred embodiment of the apparatus of the present invention in which an array of electrodes is integral with a cell culture vessel;

FIGS. 2A and 2B illustrate a schematic diagram of another preferred embodiment of the apparatus of the present invention, in which an electrode array is formed on an insulating substrate;

FIGS. 3A and 3B illustrate several ways of placing an electrode array into a cell culture vessel in accordance with the present invention;

FIG. 4 illustrates an electrode array that further contains signal generating or conditioning circuits integrated upon the same substrate;

FIG. 5 illustrates an alternate embodiment containing multiple culture vessels disposed on a common ground plane;

FIG. 6 illustrates the application of the inventive apparatus to biological samples such as seeds, protocorms, or the like;

FIG. 7 illustrates the use of a porous layer disposed upon an electrode array in order to immobilize the cells and suppress fluid convection;

FIG. 8 illustrates an alternate embodiment in which a porous substrate has a ground plane on one surface and an electrode array disposed on the opposite surface;

FIG. 9 illustrates an alternate embodiment in which the sample is exposed to a plurality of oscillating electromagnetic fields by an array of antennas disposed underneath selected areas of the sample; and

FIG. 10 illustrates one sample chamber of the device in FIG. 5 further containing an electrically conductive porous structure in contact with the ground plane.

### DESCRIPTION OF PREFERRED EMBODIMENTS

The invention and the various features and advantageous details thereof are explained more fully with reference to the nonlimiting embodiments that are illustrated in the accompanying drawings and detailed in the following description. Descriptions of well known components and processing techniques are omitted so as not to unnecessarily obscure the invention in detail. It should be understood, however, that the detailed description and the specific examples, while indicating preferred embodiments of the invention, are given by way of illustration only and not by way of limitation. Various substitutions, modifications, additions and/or rearrangements within the spirit and/or scope of the underlying inventive concept will become apparent to those skilled in the art from this detailed description.

The biological responses and phenomena can take a number of forms. A number of these effects are discussed below.

Bone growth, or osteogenesis, involves several different cell types, including osteoclasts and osteoblasts. Their behavior in the presence of stress is a significant factor in maintaining bone strength, and some possible explanations for their behavior invoke electrical signals generated by the piezoelectric nature of the mineral crystallites within the bone.

Burn healing can also be stimulated by the flow of small electric currents. The influence of polarity, frequency, etc. has not been studied at length because of the time and expense that would be involved in maintaining a large number of injured laboratory animals in order to conduct such a systematic study.

The broad topic of inflammatory response is an important object of study with respect to implants and prostheses. Thrombogenesis, for example, is a complex, multistep process that begins with attachment of proteins such as fibronectin to a foreign surface. Adhesion of platelets, macrophages, and other entities proceeds from there, eventually creating a clot, capsule, or other abnormal deposit.

The production and release of proteins, hormones, and other compounds is important not only in health or disease conditions but also in bioprocessing strategies and fermentation. Properly applied electric fields could increase the output of a bioprocessor or stimulate a more normal production of hormones from an underactive gland in the body.

The stimulation or inhibition of tissue growth has many implications in healing, scar formation, and human development. A related topic is the differentiation of embryonic cells, such as those in protocorms or blastocysts, into specific cell lines and structures.

Bacterial growth in the presence of electric fields can be important, especially as it pertains to antibiotic resistance mechanisms. Combining standard isolation and culture techniques with electric fields could identify regimens for enhancing the performance of antibiotics and would also serve as a further differentiation technique to identify and isolate new bacterial strains.

The rapidly developing field of gene expression analysis could be expanded into another dimension, wherein a particular tissue could be exposed to different electric fields while mapping the degree to which particular genes are expressed at those locations.

Because of the wide range of possible biological parameters that might be influenced by external fields, a number of different configurations of test apparatus may be appropriate. For example, in cases where cellular growth or proliferation is to be observed with an optical microscope, a transparent test enclosure, analogous to a Petri dish may be needed. This enclosure should be easily sterilized if it is to be reused like traditional laboratory ware. Conversely, if the data are collected destructively, for example, by scanning electron microscope (SEM) examination, the apparatus should employ very low-cost consumables.

It will be appreciated that the inventive apparatus may contain one or more ground planes or grounded electrodes. It will be further understood that the potential applied by any particular electrode may be a DC or AC potential with any selected waveform. The aforementioned ability to selectively bias small areas of a sample with complex waveforms will further increase the ability to discover the most suitable electrical stimulation for specific applications.

It will also be appreciated that some biological problems will involve a single sample of substantially solid tissue, while others might involve single cells, bacteria, algae, or the like. Other experiments might involve batches of more or less identical samples such as seeds, spores, blastocysts, protocorms, etc. to be subjected to different electric fields at the same time in an otherwise similar environment. Applicant's inventive combination of a uniform culture environment and selectively nonuniform electric fields can be applied equally well to all of the foregoing experimental problems by a properly configured electrode array.

An apparatus incorporating various features of the present invention is illustrated generally at 10 in the figures. The apparatus 10 is designed to allow a biological sample 11 to be maintained in a relatively uniform culture environment while an array of electrodes 12 imposes a number of selected electric fields on the sample 11.

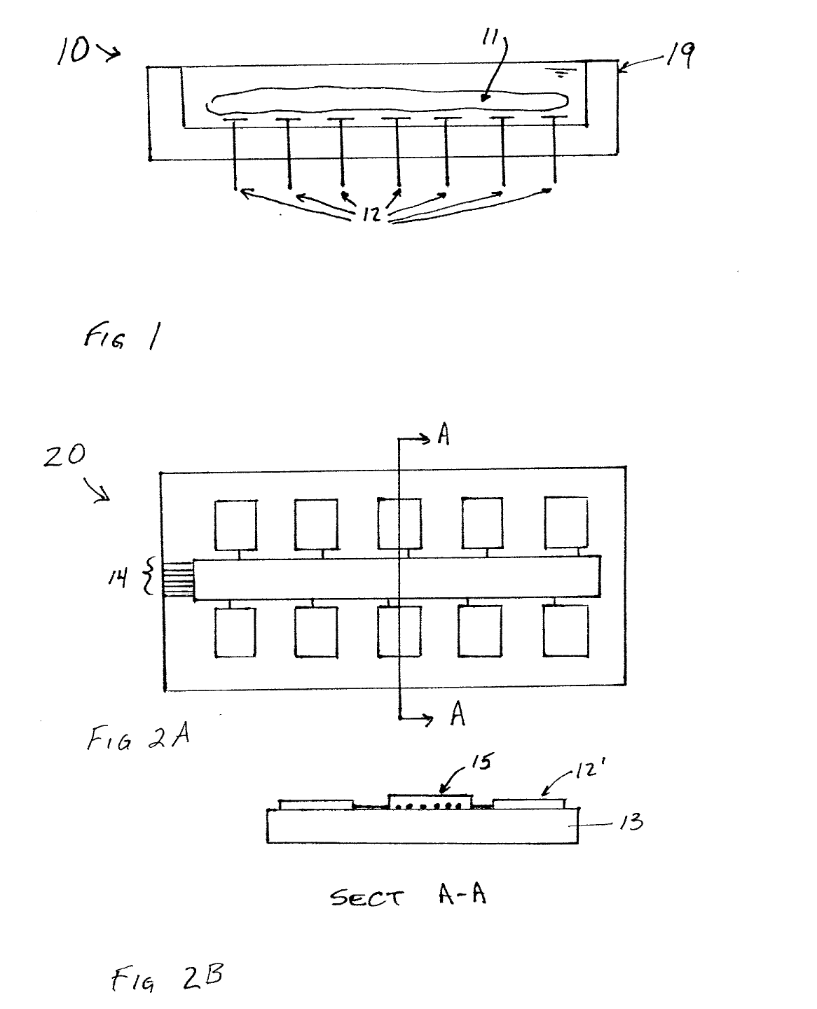

FIG. 1 illustrates schematically one preferred embodiment of the apparatus 10 of the present invention, wherein a selected sample 11 is maintained in a cavity within a container 19. The electrodes 12 penetrate the bottom of the container. The bottom of the container can be constructed from an insulating material such as glass, ceramic, plastic, or the like. The bottom of the container can include, or compose, a substrate. The term substrate as used herein is defined as a structure that physically provides at least one surface and an insulating function between the electrodes 12. Although the substrate can include a nutrient, the substrate does not need to include a nutrient. The sample 11 is cultured for some period of time while different selected electric fields are applied to the different areas via the electrodes 12. Selected biological effects are observed at these same areas, whereby the influence of electric fields may be isolated from other effects owing to the fact that the different areas of the sample 11 exist in otherwise uniform environment. Applicable processes include, but are not limited to, cell growth and healing, scar formation, osteoclast and osteoblast behavior, seed germination, bacterial growth or inhibition, production of proteins or hormones, inflammatory responses, and uptake of pharmaceuticals. It will be understood that the term “sample” as used within the present disclosure refers to a selected biological medium containing one or more cell types and any associated fluids, nutrients, or other materials necessary or incidental to the experiment. It will be further understood that the inventive device and methods may include ancillary items for the control of temperature, cover gas (aerobic or anaerobic), humidity, pH, fluid flow, etc. as are well known in the art of biological experimentation.

It will be appreciated that selected electric potentials may be established between any two adjacent electrodes 12 or between individual electrodes 12 and a common ground plane (not shown) disposed in the culture medium just above the sample 11 and in electrical contact therewith.

The array illustrated in FIG. 1 is not optimized, but rather is an illustration of the potential of the inventive device and associated method to gather a large amount of valuable information from a single biological sample. Skilled artisans will appreciate that the inventive technique could be used to create even more optimal results by, for example, including temperature monitoring and control, and electrodes specifically configured for particular samples. It will also be appreciated that the electrodes may be a bioinert material such as gold, or they may be a specific material of interest for implants, prostheses, or various other biomedical uses where a metal surface interacts with living tissue. In all of the foregoing modifications, Applicant's technique allows one to isolate and study the specific effects of different electric fields because of the uniform “baseline” culture environment within the test vessel.

For simplicity, commonly used engineering features such as connectors, external wiring, etc. are not shown in the drawings. Those skilled in the art will readily understand that a variety of electrical interconnection techniques may be used without departing from the essential features of the invention. For example, a structure similar to that shown in FIG. 1 may be constructed using multilayer ceramic packaging techniques commonly used for microprocessor chips. A ceramic package of this type would be easily mass-produced and easily sterilized for reuse. The culture vessel would therefore be a simple plug-in module. Standardized multi-pin connectors would provide a reliable interface to the power supply and any related electronic devices.

### EXAMPLES

Specific embodiments of the invention will now be further described by the following, nonlimiting examples which will serve to illustrate in some detail various features. The following examples are included to facilitate an understanding of ways in which the invention may be practiced. It should be appreciated that the examples which follow represent embodiments discovered to function well in the practice of the invention, and thus can be considered to constitute preferred modes for the practice of the invention. However, it should be appreciated that many changes can be made in the exemplary embodiments which are disclosed while still obtaining like or similar result without departing from the spirit and scope of the invention. Accordingly, the examples should not be construed as limiting the scope of the invention.

### Example 1

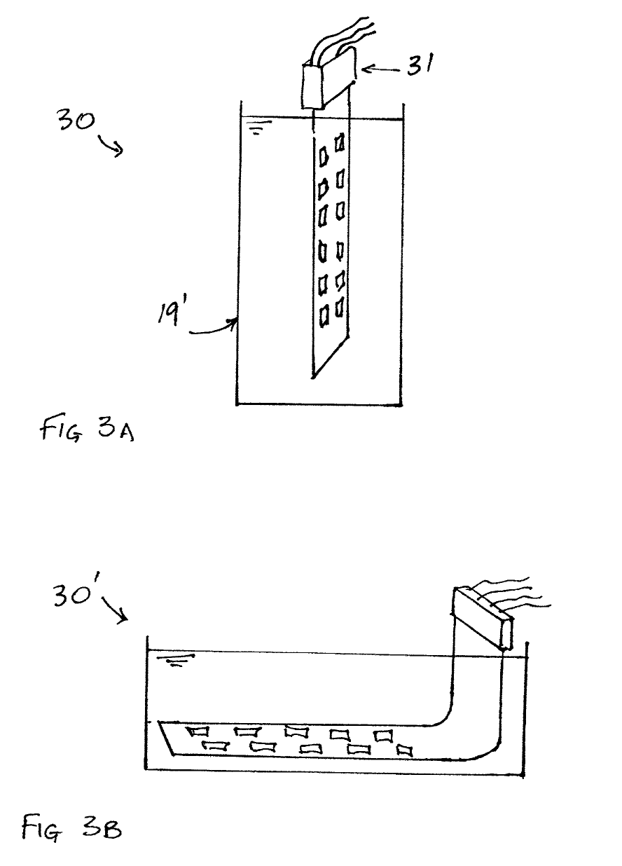

Illustrated at 20 in FIG. 2 is an alternate embodiment of the apparatus of the present invention. In this alternate embodiment, the electrodes 12′ are disposed on an insulating substrate 13 that is separate from the container 19′ as shown in FIG. 3. In this case, the lead wires 14 are disposed on the top surface of substrate 13 and covered by an insulating layer 15 as shown. Alternatively, the leads 14 may be placed on the underside of the substrate 13 and connected to the electrodes 12′ by means of vias, plated through holes, or any similar arrangement. Either of the foregoing structures will be familiar to those skilled in the art of thick-film, thin-film, or printed circuit design.

It will be appreciated that the electrode array 20 shown in FIG. 2 may be disposed on a thin, flexible substrate such as polyimide or the like. A conventional cable connector 31 may be attached to one end of the substrate 13 and the array may be deployed either vertically or horizontally within a culture vessel 19′ as shown respectively at 30 and 30′.

### Example 2

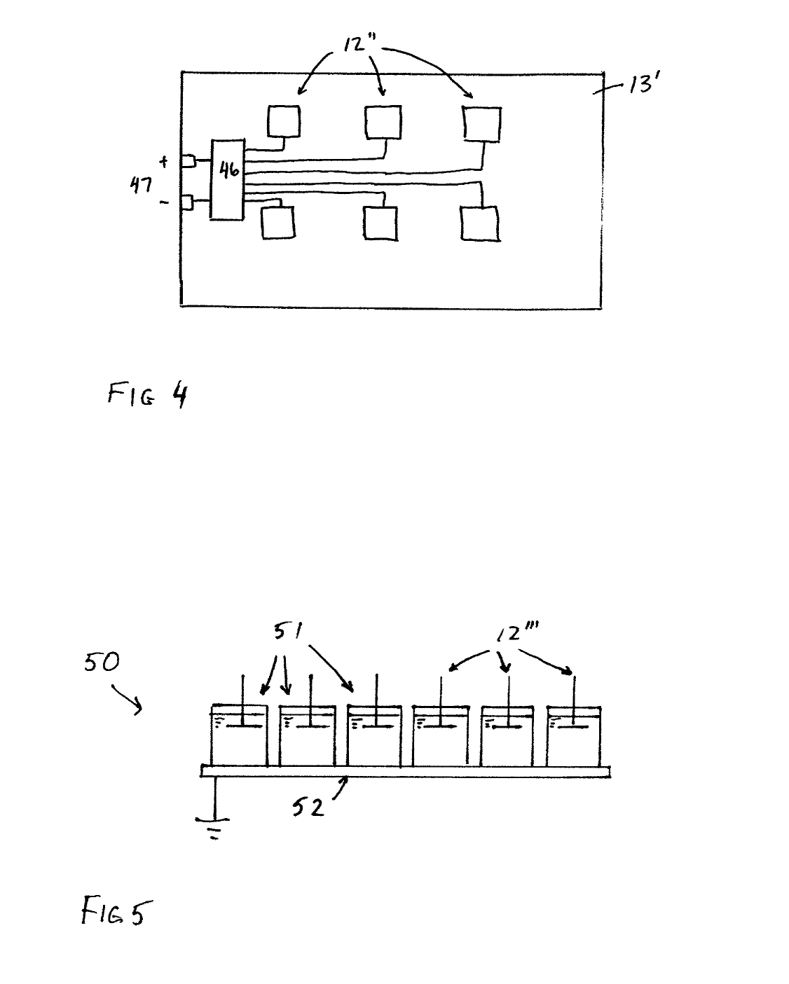

The electrode arrays shown in FIGS. 1-3 are completely passive devices, relying on an external power supply, signal generator, or the like (not shown) to produce the desired potentials at each electrode 12. In a system similar to that in the preceding example, FIG. 4, an active circuit 46 accepts input power at 47 from an external power supply and generates the desired DC or AC signals for each of the electrodes 12″. It will be appreciated that the circuit 46 may be a single silicon die attached to the substrate 13′ by conventional wire-bond or flip-chip techniques and passivated by a glob-top or die-underfill material. Alternatively, the circuit 46 may be a hybrid circuit, multichip module or even a plurality of individual circuits serving the individual electrodes. Furthermore, the circuit 46 could contain a battery, thereby obviating the need for an external power supply.

### Example 3

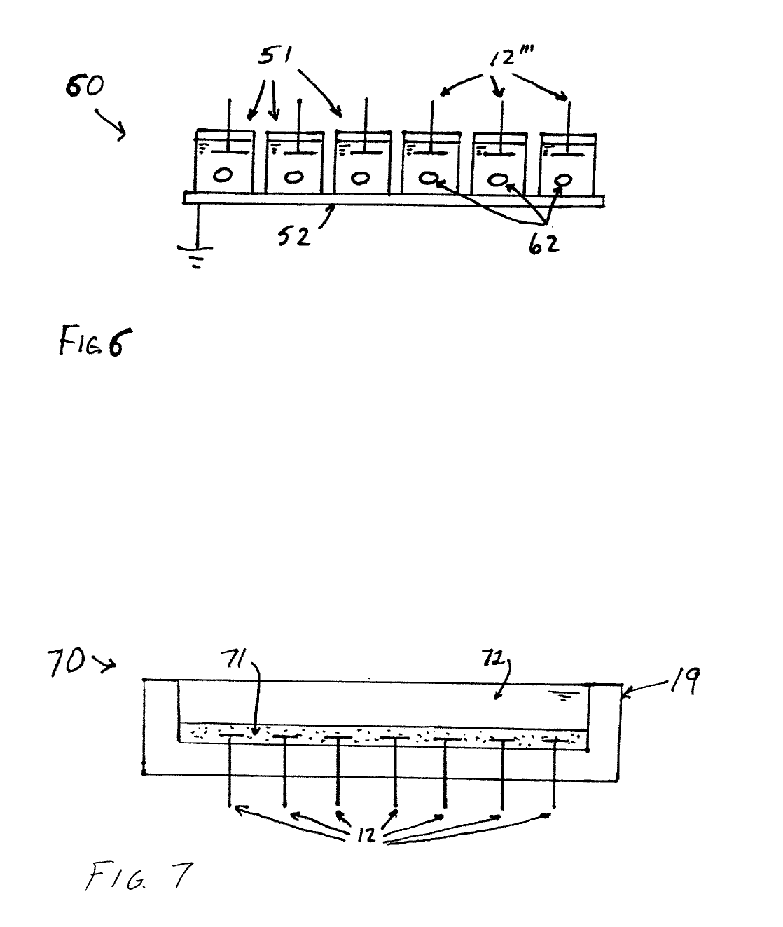

In cases where it is desirable to keep individual samples isolated from one another, the inventive technique can be modified as shown at 50 in FIG. 5. Here, a number of individual sample compartments 51 are arranged on a common ground plane 52. The electrodes 12′″ are placed in the top of the compartments 51 thereby establishing selected potentials with respect to the ground plane 52. It will be appreciated that while the Figure illustrates the compartments 51 as separate tubes or vials, this arrangement can equally well be achieved using a nonconductive latticework of polymer or the like forming partitions that can be sealed against the ground plane 52 and divide its area into any number of (typically square) subdivisions.

A rectangular array of individual compartments as described in the foregoing Example allows one to conduct factorial experiments of various types. For instance, in a square array each row could represent one electric field and each column could represent one concentration of a particular nutrient or antibiotic. Again, other than this additional variable, the samples will experience substantially uniform environmental conditions, allowing the researcher to elucidate the effects of the particular experimental variables.

### Example 4

The terms “cells,” “tissues,” and “biological samples” must be read to include such recognizable biological structures as seeds, protocorms, spores, blastocysts, or eggs where the effect of electric fields on survival, germination, tissue differentiation, etc. is to be studied. FIG. 6 shows an apparatus like that in FIG. 5, but in this case each compartment 51′ contains a single object 52 such as a seed or protocorm.

The inventive device can be used with substantially solid tissue samples as well as substantially liquid samples (blood, body fluids, etc.) in which the motion or attachment of cells, platelets, proteins, etc. might be the parameter of interest. Conversely, it will be desirable in some cases to provide a means of immobilizing the cells, suppressing fluid convection, or otherwise ensuring that any particular cell remains in the selected electric field region. For these situations, a porous coating can be placed on the array and inoculated with the cells.

### Example 5

Illustrated at 70 in FIG. 7 is an array of electrodes 12 covered with a porous layer 71 in which the cells are immobilized for study. A fluid medium, possibly containing nutrients, 72 may be provided. Alternatively, nutrients may be incorporated directly into the layer 71, analogous to products such as those typified by 3M™ Petrifilm™ Aerobic Count Plates (3M Microbiology Products, St. Paul, Minn.), requiring only the addition of water to prepare the layer 71 for inoculation with the cells to be studied. In this case, the electrodes and their circuit lines would be formed on an insulating substrate such as polyimide by photolithography, printing, or the like. Insulation would be printed over the circuit lines, and then the culture medium would be deposited upon the entire surface, thereby allowing the electrodes 12 to maintain intimate contact with the layer 71. The entire assembly may be stored dry and moistened prior to activation. The porous layer 71 may be a layer of gelatin or agar, a cross-linked hydrogel, a porous inorganic layer, low-density polyethylene (LDPE), or other selected material having appropriate biocompatibility with the cells to be studied.

It will be appreciated that the porous layer 71 in the foregoing Example may incorporate other selected chemical constituents to render the inventive apparatus and method even more useful. For example, antibiotic compounds may be added in order to study the effects of electric fields on antibiotic resistance. Stain chemistries may be incorporated to allow for the ready visualization of bacterial colonies or the like. It will be further appreciated that several cell types may be present, as for example, to study the interactions of electric fields with the response of macrophages to pathogenic bacterial cells.

### Example 6

An alternative configuration, illustrated at 80 in FIG. 8, comprises a thin slab of porous material 81 such as LDPE into which the cells and culture medium are inoculated. Electrodes 12 (only one of which is shown for simplicity) are disposed on one surface. A ground plane 82 is disposed on the opposite surface. The connecting circuit lines 14′ are isolated by layers of insulating dielectric material 15′, 15″ above and below in order to localize the desired electric field at the electrodes 12. This embodiment would be especially useful as a disposable electrode array for routine use.

### Example 7

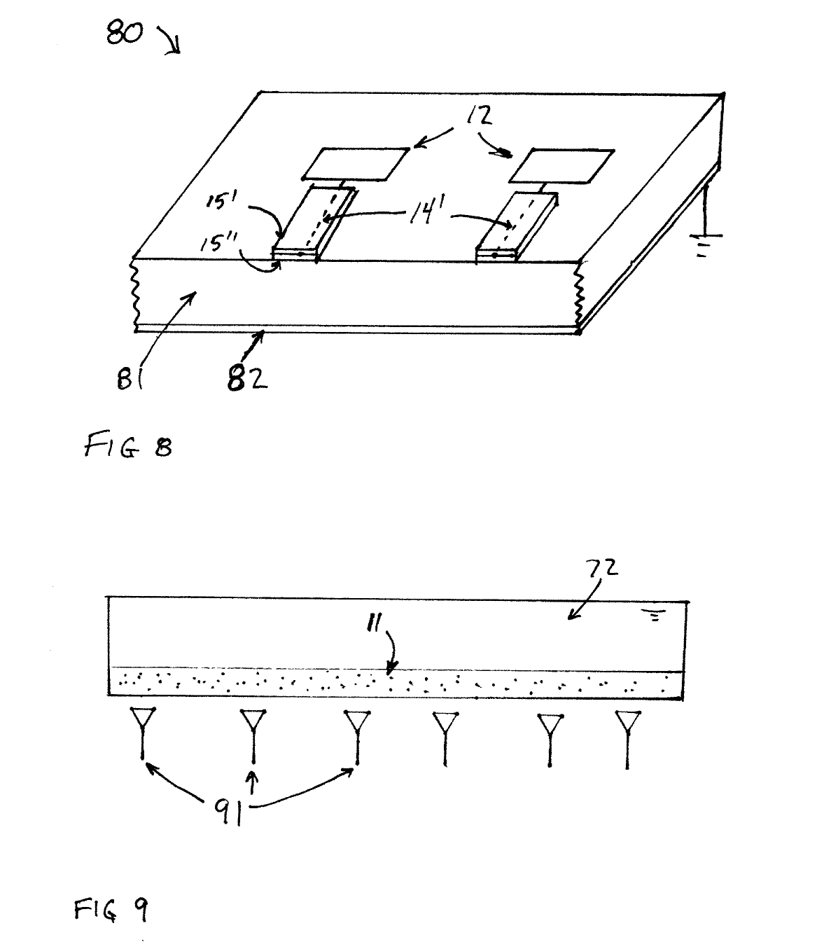

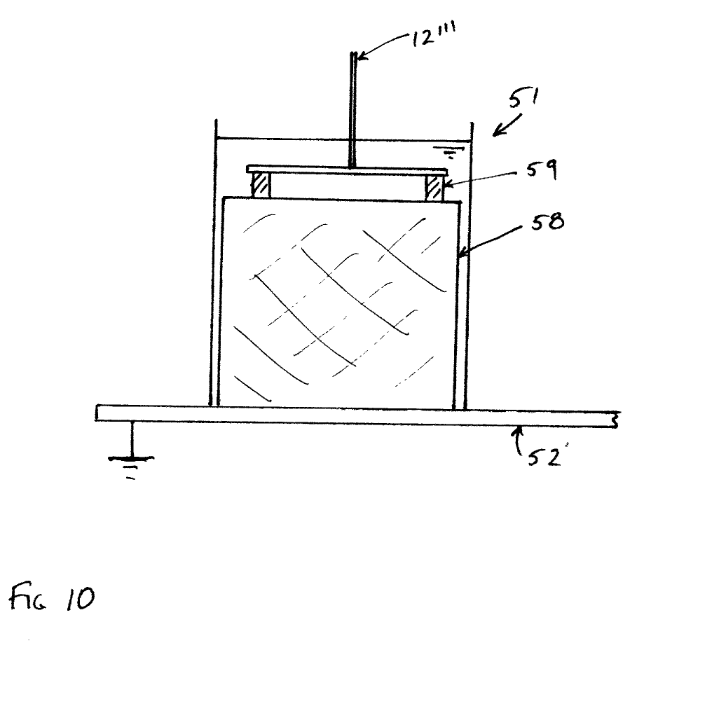

As noted above, both DC and AC fields may be of interest in particular applications. It will be understood that chopped DC, sine wave, square-wave, or virtually any desired waveform may be useful for particular situations. The previous examples illustrated devices in which the electrodes 12 are in direct electrical contact with the tissue or cell culture medium. Direct electrical contact is most convenient for maintaining a desired DC field at a desired point on the sample. However, it will be appreciated that pulsed DC or AC fields, and especially electromagnetic fields, may alternatively be coupled to the sample inductively by means of small antennas 91 shown in FIG. 9 or capacitively by means of planar electrodes disposed beneath a thin dielectric layer.

### Example 8

The device shown in Example 3 can also be used to elucidate the effects of electric field on the production of various chemical entities from living samples. In this case, the individual sample chambers serve to isolate the products in the various chambers so that these may be sampled (by means of a pipette, for instance). It will be readily appreciated that the inventive technique may be combined with high-throughput gene expression analysis as is well known in the art. In this way, a comprehensive library of data may be generated for all manner of tissue types, that would indicate how or if the application of particular electric fields influences the expression of particular genes. It will be further appreciated that the individual chambers may be temporarily hermetically sealed, whereby evolving gaseous products within each chamber may be trapped for analysis and comparison to those in other chambers.

### Example 9

Another useful application of the inventive technique described in the preceding Example relates to the development of improved bioprocessing strategies. It is well known that applying an electrical potential to bacteria can in some cases enhance the production of various fermentation products or other metabolites. By culturing an array of similar samples while keeping the liquid medium in each chamber separate, one can quantify the production of the chemical species of interest in each chamber and thereby quantify the effect of imposed electrical potentials on bioprocessing efficiency. For these experiments, a further modification of the inventive device, FIG. 10, is the addition of a porous or high surface-area conductive structure 58. This structure would be colonized by the bacteria being studied and provide a convenient means of imposing the desired electric field on the bacteria. Suitable materials for this purpose include carbon fiber composites, carbon felt, and carbon foam as well as other porous conductive materials. It will be understood that the aforementioned porous material will be placed into electrical contact with either the ground plane or the active electrode in each chamber, but not both.

### Example 10

In the modified device described in the previous Example, the porous conductive material 58 is affixed to one of the two electrodes in each chamber. However, for some applications it might further be useful to remove the porous material as part of the experiment. An example would be to determine total cell mass or other characteristics that are best determined by direct examination of the biofilm on the porous material. In this case, the material is preferably made from a low-density carbon-bonded carbon-fiber composite [G. C. Wei and J. M. Robbins, Carbon Bonded Carbon-Fiber Insulation for Radioisotope Space Power Systems, Cer. Bull. 64[5], 1985] in the form of a pellet. These pellets may be connected to the upper electrodes or they may be placed into the chambers in contact with the lower electrode (ground plane) as shown in FIG. 10, in which case a small insulating spacer 59 of polymer, ceramic, or the like may be placed between the porous pellet and the upper electrode. This arrangement assures good electrical contact with the ground plane 52 as well as electrical separation from the upper electrode 12′″.

While several preferred embodiments of the inventive apparatus have been shown and described, and several embodiments of the inventive method have been specifically delineated, it will be understood that such descriptions are not intended to limit the disclosure, but rather it is intended to cover all modifications and alternate methods falling within the spirit and the scope of the invention as defined in the appended claims or their equivalents.

All the disclosed embodiments of the invention disclosed herein can be made and used without undue experimentation in light of the disclosure. Although the best mode of carrying out the invention contemplated by the inventor(s) is disclosed, practice of the invention is not limited thereto. Accordingly, it will be appreciated by those skilled in the art that the invention may be practiced otherwise than as specifically described herein.

The individual components need not be formed in the disclosed shapes, or combined in the disclosed configurations, but could be provided in virtually any shapes, and/or combined in virtually any configuration. Further, the individual components need not be fabricated from the disclosed materials, but could be fabricated from virtually any suitable materials. Further, homologous replacements may be substituted for the substances described herein. Further, agents which are both chemically and physiologically related may be substituted for the agents described herein while the same or similar results would be achieved. Further, variation may be made in the steps or in the sequence of steps composing methods described herein.

Further, although the individually addressable electrodes described herein can be a separate module, it will be manifest that the individually addressable electrodes may be integrated into the system with which they are associated. Furthermore, all the disclosed elements and features of each disclosed embodiment can be combined with, or substituted for, the disclosed elements and features of every other disclosed embodiment except where such elements or features are mutually exclusive.

It will be manifest that various substitutions, modifications, additions and/or rearrangements of the features of the invention may be made without deviating from the spirit and/or scope of the underlying inventive concept. It is deemed that the spirit and/or scope of the underlying inventive concept as defined by the appended claims and their equivalents cover all such substitutions, modifications, additions and/or rearrangements.

The appended claims are not to be interpreted as including means-plus-function limitations, unless such a limitation is explicitly recited in a given claim using the phrase(s) “means for” and/or “step for.” Subgeneric embodiments of the invention are delineated by the appended independent claims and their equivalents. Specific embodiments of the invention are differentiated by the appended dependent claims and their equivalents.

What is claimed is:

1. An apparatus for screening biological samples for their response to electric fields, comprising:
a container suitable for containing a living biological sample and any associated fluids;
a substrate disposed within the container;
a plurality of independently-addressable electrodes disposed upon the substrate; and
a means of applying differing electrical potentials to the plurality of independently-addressable electrodes for a period of time, wherein different regions of the living biological sample can be cultured in the presence of different selected electric fields and the effects of those different electric fields on a biological function of the living biological sample may be determined.

2. The apparatus of claim 1 wherein the substrate includes the plurality of independently-addressable electrodes and is integral with a surface of the container.

3. The apparatus of claim 1 wherein the substrate includes the electrodes and composes a separate structure disposed within the container.

4. The apparatus of claim 1 further comprising a conductive surface that can be held at ground potential, wherein the plurality of independently-addressable electrodes may be maintained at a plurality of potentials relative to the conductive surface.

5. The apparatus of claim 1 wherein at least one surface of the container is substantially transparent.

6. The apparatus of claim 3 wherein the means of applying differing electrical potentials includes at least one active circuit element disposed upon the substrate and connected to at least one of the plurality of independently-addressable electrodes, whereby the electrical potential of the at least one of the plurality of independently-addressable electrodes may be maintained by the active circuit element.

7. The apparatus of claim 1 further comprising a porous layer disposed proximate to the plurality of independently-addressable electrodes, whereby cells may be immobilized in selected locations relative to the plurality of independently-addressable electrodes during the period of time.

8. The apparatus of claim 7 wherein the porous layer includes a material selected from the group consisting of agar, gelatin, cross-linked hydrogels, low-density polyethylene, and porous ceramics.

9. The apparatus of claim 1 wherein the container includes a plurality of individual chambers and the means of applying includes means for maintaining an independently definable electric field in each of the plurality of individual chambers, whereby contents of each of the plurality of individual chambers may be kept separate from one another.

10. The apparatus of claim 9 wherein the plurality of individual chambers are disposed upon a common ground plane and each of the plurality of individual chambers contains an electrode at an independently-definable potential relative to the common ground plane.

11. The apparatus of claim 9 further comprising different contents in at least some of the individual chambers, wherein at least one experimental parameter in addition to electric field may be varied while holding other environmental conditions substantially constant.

12. The apparatus of claim 3 wherein the substrate includes a porous material having a ground plane disposed upon one surface, the plurality of individually-addressable electrodes disposed upon an opposite surface, and the living biological sample is contained within the porous material.

13. The apparatus of claim 1 wherein those different electrical potentials are selected from the group consisting of continuous DC, pulsed DC, and AC.

14. The apparatus of claim 13 wherein the pulsed DC potential has a waveform defining temporal variation.

15. The apparatus of claim 13 wherein the AC potential is represented by a function selected from the group consisting of a sine wave and a square wave

16. The apparatus of claim 1 wherein the biological function is selected from the group consisting of: cell growth, proliferation and/or inhibition; platelet adhesion on a surface; gene expression; protein and/or hormone production; cell mobility and/or alignment; healing and/or scar formation; osteoclast, osteoblast and/or macrophage behavior; germination of seeds and/or spores; bacterial growth and/or inhibition; inflammatory responses; release of cell metabolites; and uptake of pharmaceuticals.

17. An apparatus for rapidly screening biological samples for their response to electric fields, comprising:
a container suitable for containing a living biological sample and associated fluids;
a substrate disposed within the container, the substrate including a plurality of independently-addressable electrodes; and
a means of applying differing electrical potentials to the plurality of independently-addressable electrodes for a period of time, wherein different regions of the living biological sample can be cultured in the presence of different electric fields while maintaining other environmental parameters substantially constant and the effects of those different electric fields on at least one biological function of the living biological sample may be determined.

18. The apparatus of claim 17 wherein the substrate is integral with a surface of the container.

19. The apparatus of claim 17 wherein the substrate composes a separate structure disposed within the container.

20. The apparatus of claim 17 further comprising a conductive surface at ground potential, wherein the plurality of independently-addressable electrodes may be maintained at a plurality of potentials relative to the conductive surface.

21. The apparatus of claim 17 wherein at least one surface of the container is substantially transparent.

22. The apparatus of claim 17 wherein the means of applying differing electrical potentials includes at least one active circuit element disposed upon the substrate and connected to at least one of the plurality of independently-addressable electrodes, whereby the electrical potential of the at least one of the plurality of independently-addressable electrodes may be maintained by the active circuit element.

23. The apparatus of claim 17 further comprising a porous layer disposed proximate to the plurality of independently-addressable electrodes, whereby cells may be immobilized in selected locations relative to the plurality of independently-addressable electrodes during the period of time.

24. The apparatus of claim 23 wherein the porous layer includes a material selected from the group consisting of agar, gelatin, cross-linked hydrogels, low-density polyethylene, and porous ceramics.

25. The apparatus of claim 17 wherein the container includes a plurality of individual chambers and the means of applying includes means for maintaining an independently-definable electric field in each of the plurality of individual chambers, whereby the contents of each of the plurality of individual chambers may be kept separate from one another.

26. The apparatus of claim 25 wherein the plurality of individual chambers are disposed upon a common ground plane and each of the plurality of individual chambers contains an electrode at an independently-definable potential relative to the common ground plane.

27. The apparatus of claim 25 further comprising different contents in at least some of the individual chambers, wherein at least one experimental parameter in addition to electric field may be varied while holding other environmental conditions substantially constant.

28. The apparatus of claim 19 wherein the substrate includes a porous material having a ground plane disposed upon one surface, the plurality of independently-addressable electrodes disposed upon an opposite surface, and the living biological sample is contained within the porous material.

29. The apparatus of claim 17 wherein those different electrical potentials are selected from the group consisting of continuous DC, pulsed DC, and AC.

30. The apparatus of claim 29 wherein the pulsed DC potential has a selected waveform defining temporal variation.

31. The apparatus of claim 29 wherein the AC potential is represented by a function selected from the group consisting of a sine wave, and a square wave.

32. The apparatus of claim 17 wherein the biological function is selected from the group consisting of: cell growth, proliferation and/or inhibition; platelet adhesion on a surface; gene expression; protein and/or hormone production; cell mobility and/or alignment; healing and/or scar formation; osteoclast, osteoblast and/or macrophage behavior; germination of seeds and/or spores; bacterial growth and/or inhibition; inflammatory responses; release of cell metabolites; and uptake of pharmaceuticals.

33. A method for determining an optimal electric field for enhancing a biological function comprising:
placing a living biological sample and optional associated fluids into a container, the container including a substrate having thereon a plurality of independently-addressable electrodes;
applying independently-definable electrical potentials to the plurality of independently-addressable electrodes over a period of time, wherein areas of the living biological sample are exposed to electric fields while being exposed to otherwise substantially similar environmental conditions; and
examining the living biological sample to assess effects of those electric fields on a biological function of the living biological sample, wherein the optimal electric field may be systematically determined.

34. The method of claim 33 wherein the plurality of independently-addressable electrodes maintain substantially ohmic contact with the living biological sample.

35. The method of claim 33 wherein the plurality of independently-addressable electrodes are substantially not in ohmic contact with the living biological sample and those electric fields are capacitively coupled to the living biological sample.

36. The method of claim 33 wherein the plurality of independently-addressable electrodes are substantially not in ohmic contact with the living biological sample and those electric fields are inductively coupled to the living biological sample.

37. The method of claim 33 wherein at least one surface of the container is substantially transparent and the living biological sample may be visually examined during a period of time during which the living biological sample is exposed.

38. The method of claim 37 wherein visual examination is performed with an optical microscope.

39. The method of claim 33 further comprising utilizing a porous layer disposed proximate to the electrodes to immobilize cells in locations relative to the plurality of independently-addressable electrodes during the period of time.

40. The method of claim 39 wherein the porous layer includes a material selected from the group consisting of agar, gelatin, cross-linked hydrogels, low-density polyethylene, and porous ceramics.

41. The method of claim 39 wherein the porous layer includes a chemical staining agent and colonies of those cells may be more readily visible.

42. The method of claim 33 wherein the container includes a plurality of individual chambers and means for maintaining an independently-definable electric field in each of the chambers and contents of each of the plurality of individual chambers may be kept separate from one another.

43. The method of claim 42 wherein the contents of at least some of the plurality of individual chambers are different and at least one experimental parameter in addition to electric field may be varied while holding other environmental conditions substantially constant.

44. The method of claim 43 wherein the living biological sample includes pathogenic bacteria and the plurality of individual chambers contain selected concentrations of at least one antibiotic compound.

45. The method of claim 33 wherein the independently-definable electrical potentials are selected from the group consisting of continuous DC, pulsed DC, and AC.

46. The method of claim 42 wherein each of the plurality of individual chambers includes a porous, electrically conductive structure in electrical contact with one of the plurality of independently-addressable electrodes and those electric fields may be applied to a film of bacteria growing on the porous, electrically conductive structure.

47. The method of claim 33 wherein the selected biological function is selected from the group consisting of: cell growth, proliferation and/or inhibition; platelet adhesion on a surface; gene expression; protein and/or hormone production; cell mobility and/or alignment; healing and/or scar formation; osteoclast, osteoblast and/or macrophage behavior; germination of seeds and/or spores; bacterial growth and/or inhibition; inflammatory responses; release of cell metabolites; and uptake of pharmaceuticals.

48. A method for determining an optimal electric field for enhancing a selected biological process comprising:
placing a living biological sample and optional associated fluids into a container, the container including an electrically insulating structure having thereon a plurality of independently-addressable electrodes;
applying independently-definable electrical potentials to the plurality of independently-addressable electrodes over a period of time; and
examining the living biological sample to assess effects of those electric fields on a biological function of the living biological sample, wherein the optimal electric field may be rapidly determined.

49. The method of claim 48 wherein the plurality of independently-addressable electrodes maintain a substantially ohmic contact with the living biological sample.

50. The method of claim 48 wherein the plurality of independently-addressable electrodes are substantially not in ohmic contact with the living biological sample and those electric fields are capacitively coupled to the living biological sample.

51. The method of claim 48 wherein the plurality of independently-addressable electrodes are substantially not in ohmic contact with the living biological sample and those electric fields are inductively coupled to the living biological sample.

52. The method of claim 48 wherein at least one surface of the container is substantially transparent and the living biological sample may be visually examined during a time period during which the living biological sample is exposed.

53. The method of claim 52 wherein visual examination is performed with an optical microscope.

54. The method of claim 48 further comprising utilizing a porous layer disposed proximate to the electrodes to immobilize cells in locations relative to the plurality of independently-addressable electrodes during the period of time.

55. The method of claim 54 wherein the porous layer includes a material selected from the group consisting of agar, gelatin, cross-linked hydrogels, low-density polyethylene, and porous ceramics.

56. The method of claim 54 wherein the porous layer includes a chemical staining agent and colonies of those cells may be more readily visible.

57. The method of claim 48 wherein the container includes a plurality of individual chambers and means for maintaining an independently-definable electric field in each of the chambers and contents of each of the plurality of individual chambers may be kept separate from one another.

58. The method of claim 57 wherein the contents of at least some of the plurality of individual chambers are different and at least one experimental parameter in addition to electric field may be varied while holding other environmental conditions substantially constant.

59. The method of claim 57 wherein the living biological sample includes pathogenic bacteria and the plurality of individual chambers contain selected concentrations of at least one antibiotic compound.

60. The method of claim 48 wherein the independently-definable electrical potentials are selected from the group consisting of continuous DC, pulsed DC, and AC.

61. The method of claim 57 wherein each of the individual chambers includes a porous, electrically conductive structure in electrical contact with one of the plurality of independently-addressable electrodes and those electric fields may be applied to a film of bacteria growing on the porous, electrically conductive structure.

62. The method of claim 48 wherein the selected biological function is selected from the group consisting of: cell growth, proliferation and/or inhibition; platelet adhesion on a surface; gene expression; protein and/or hormone production; cell mobility and/or alignment; healing and/or scar formation; osteoclast, osteoblast and/or macrophage behavior; germination of seeds and/or spores; bacterial growth and/or inhibition; inflammatory responses; release of cell metabolites; and uptake of pharmaceuticals.
